(19) United States
(12) Patent Application Publication
(10) Pub. No.: US 2010/0174238 A1
(43) Pub. Date: Jul. 8, 2010

(54) INFUSION AND SUCTION CONFIGURATION IN AN ATHERECTOMY SYSTEM

(75) Inventor: Arieh Sher, Rehovot (IL)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(73) Assignee: ArioMedica Ltd., Tel Aviv (IL)

(21) Appl. No.: 12/665,719

(22) PCT Filed: Jun. 12, 2008
(86) PCT No.: PCT/IL08/00806

§ 371 Date: Dec. 20, 2009

Related U.S. Application Data
(60) Provisional application No. 60/945,355, filed on Jun. 21, 2007.

(51) Int. Cl. A61M 37/00; A61B 17/22

(52) U.S. Cl. 604/131; 606/159

(57) ABSTRACT

A method for expelling blood and preventing blood from entering portions of an atherectomy system that includes the passage of infusion fluid through a passageway configured in the atherectomy system so as to provide a flow of infusion fluid through an operational mechanism of the atherectomy system and exit the atherectomy system at a higher pressure and higher velocity than that of the blood flow within the blood vessel in which the atherectomy system is operating. The method includes lubricating portions of the atherectomy system by bringing infusion fluid into contact with components of the operational mechanism of the atherectomy system, wherein a relative position of the two components one to another varies during operation. The atherectomy system includes a control system that monitors and regulates the amount of fluid infused into, and the fluid leaving, the artery by a weight comparison method in which the relative weight difference between the infusion bag and the effluent collecting bottle is compared.

### FIELD AND BACKGROUND OF THE INVENTION

The present invention relates to infusion and suction configurations for use in atherectomy systems and, in particular, it concerns an infusion and suction configuration for use in an atherectomy system in which debris is flushed from portions of the operational mechanism of the atherectomy system, such mechanism is also lubricated by the infusion fluid and the flow of fluid infused into the artery and the flow of fluid leaving the artery is monitored and regulated by a weight method.

Systems that use infusion and vacuum aspiration simultaneously are known in the art. Representative of these systems are U.S. Pat. No. 6,818,001 to Wulfman et al. which describes an atherectomy device. Other examples are U.S. Pat. No. 6,129,697 to Drasler et al. and Us Patent Application 20070073233 to Thor et al., which describes a thrombectomy system that achieves iso-volumetric balance between fluid delivery and fluid removal.

There is therefore a need for an infusion and suction configuration for use in an atherectomy system in which debris is flushed from portions of the operational mechanism of the atherectomy system, such mechanism is also lubricated by the infusion fluid and the flow of fluid infused into the artery and the flow of fluid leaving the artery is monitored and regulated by a weight method.

### SUMMARY OF THE INVENTION

The present invention is an infusion and suction configuration for use in an atherectomy system in which debris is flushed from portions of the operational mechanism of the atherectomy system, such mechanism is also lubricated by the infusion fluid and the flow of fluid infused into the artery and the flow of fluid leaving the artery is monitored and regulated by a weight method.

According to the teachings of the present invention there is provided, a method for expelling blood and preventing blood from entering portions of an atherectomy system, the method comprising the passage of infusion fluid through a passageway configured in the atherectomy system, said passageway configured to provide a flow of said infusion fluid through an operational mechanism of the atherectomy system and exit the atherectomy system at a higher pressure and higher velocity than that of blood flow within a blood vessel in which the atherectomy system is being operated.

According to a further teaching of the present invention, there is also provided implementing said passageway between an inside wall of a stationary outer tube and an outside wall of an inner displaceable tube.

According to a further teaching of the present invention, there is also provided directing said infusion fluid through at least a portion of a rotation generating arrangement of the atherectomy system.

According to a further teaching of the present invention, there is also provided lubricating portions of the atherectomy system, by bringing said infusion fluid into contact with at least two components of an operational mechanism of the atherectomy, system, wherein a relative position of said at least two components one to another is varied during operation of the atherectomy system.

There is also provided according to the teachings of the present invention, an atherectomy system comprising a passageway configured to bring an infusion fluid into contact with at least a two components of an operational mechanism of the atherectomy system, wherein a relative position of said at least two components one to another is varied during operation of the atherectomy system.

According to a further teaching of the present invention, said at least two components are substantially adjacent one to another.

According to a further teaching of the present invention, said passageway is configured between an inside wall of a stationary outer tube and an outside wall of an inner displaceable tube.

According to a further teaching of the present invention, said passageway is configured so as to direct said infusion fluid through at least a portion of a rotation generating arrangement of the atherectomy system.

According to a further teaching of the present invention, there is also provided a pump arrangement configured to provide a fluid flow through said passageway at a higher pressure and higher velocity than that of blood within a blood vessel in which the atherectomy system is being operated so as to expel blood from said operational mechanism by.

There is also provided according to the teachings of the present invention, an atherectomy system comprising a control system that monitors and regulates a flow of fluid infused into an blood vessel and a flow of fluid leaving said blood vessel by a weight comparison method in which a relative weight difference between an infusion bag and an effluent collecting bottle are compared.

According to a further teaching of the present invention, there is also provided at least a first load cell from which said infusion bag is suspended and at least a second load cell upon which said effluent collecting bottle is mounted.

According to a further teaching of the present invention, there is also provided at lease a first pumping arrangement for introducing infusion fluid into the atherectomy system and at least a second pumping arrangement for evacuate of a mixture of debris, blood and infusion fluid from the atherectomy system, wherein said first and second pumping arrangements are regulated by said control system based on said relative weight difference between said infusion bag and said effluent collecting bottle.

### BRIEF DESCRIPTION OF THE DRAWINGS

The invention is herein described, by way of example only, with reference to the accompanying drawings, wherein:

FIG. 1 is an isometric view of ARIO catheter;

FIG. 2 is a longitudinal cross sectional view of ARIO's proximal end;

FIG. 2a is a detail of the distal part of the proximal end of ARIO;

FIG. 3 is a longitudinal cross sectional view of ARIO's distal end; and

FIG. 4 is a schematic diagram of ARIO's infusion and suction system.

### DESCRIPTION OF THE PREFERRED EMBODIMENTS

The present invention is an infusion and suction configuration for use in an atherectomy system in which debris is flushed from portions of the operational mechanism of the atherectomy system, such mechanism is also lubricated by the infusion fluid and the flow of fluid infused into the artery and the flow of fluid leaving the artery is monitored and regulated by a weight method.

The principles and operation of an infusion and suction configuration for use in an atherectomy system according to the present invention may be better understood with reference to the drawings and the accompanying description.

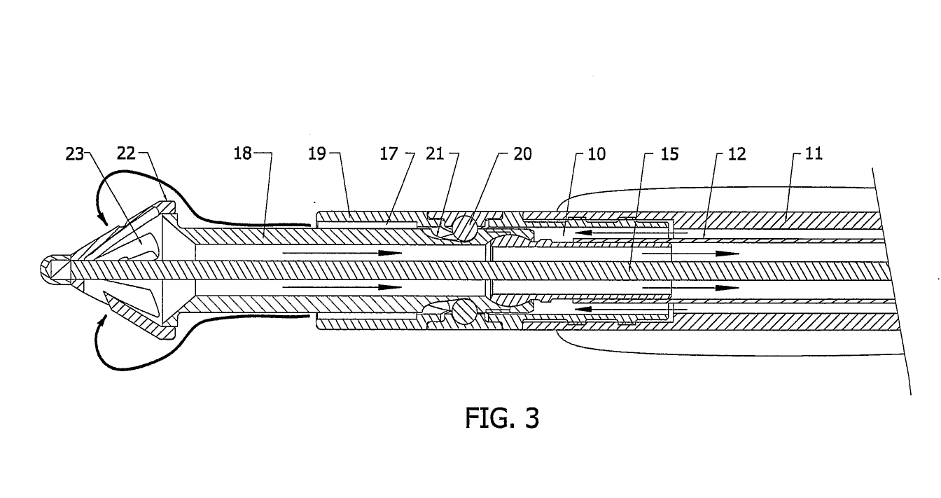

By way of introduction, the present invention is directed to improvement of the infusion and suction method and mechanism in the ARIO (Apparatus for Removal of Intraluminal Occlusions) atherectomy system. My PCT application IL2003/00807, the disclosure of which is incorporated herein by reference in its entirety, describes infusion of therapeutic liquid to the site of atheroma via a lumen that is located in the outer tube (See PCT application IL2003/00807 FIG. 3—reference number 26). The outer tube is a multi-lumen that, in addition to the infusion lumen, contains also three (or six) lumens for inflating three (or six) balloons. Debris is removed by a vacuum pump via an inner tube.

A control system automates set ups and monitors the amount of therapeutic liquid (usually saline or saline with anti-coagulant drugs) infused to the atheroma site and the amount of liquid leaving the body (a mixture of blood, therapeutic liquid and debris). It is important that the amount of therapeutic liquid that enters the artery by infusion and the liquid leaving the artery by vacuum are equal. Applying only vacuum to evacuate debris may cause the artery wall to collapse inward thus preventing blood flow. More dangerously, under such circumstances the cutter may excise the artery wall and cause vessel perforation. Infusion of therapeutic liquid to the atheroma site prevents collapse of the artery. The therapeutic liquid may have another objective. It is possible to choose a therapeutic liquid that can ease the trauma of excision, and/or help heal the wound and/or prevent formation of blood clot and/or lubricate moving parts and/or reduce inflammation and the rate of restenosis.

The present invention includes two new features that are described herein with regard to the ARIO system, but may be used to benefit in conjunction with other atherectomy systems. Among the objectives of these features are preventing blood from entering the ARIO system's operational mechanism such as, but not limited to, the rotation generating arrangement. A further objective is lubrication of the operational mechanism.

The first feature relates to the lumen through which infusion liquid is supplied to the site of atheroma. In PCT application IL2003/U0807 the infusion is via a lumen located in the outer tube. In the present invention liquid is transferred to the atheroma site via the concentric passage created between the inside diameter of the stationary outer tube and the outside diameter of the reciprocating inner tube. Debris is aspirated by vacuum via the inner tube. In the present invention, injecting the infusion liquid via the concentric lumen rather than the lumen in the outer tube has several advantages.

A first advantage of this first feature is that the infusion liquid is used as a technique to prevent blood from penetrating ARIO's mechanism. Blood entering into the mechanism and clinging to the surfaces of ARIO's moving parts may interfere with ARIO's operation or even totally stop its operation. The liquid that flows distally in the concentric lumen eventually passes through ARIO's mechanism. That is to say, the passage of the infusion fluid through the passageway configured in the ARIO system provides a flow of infusion fluid through at least a portion of the operational mechanism and exits the ARIO system at a higher pressure and higher velocity than that of blood flow within the blood vessel in which the ARIO system is being operated. The flow is at a higher pressure and higher velocity than that of the blood, thus expelling the blood from the mechanism. It is to be noted that in order to reduce the coagulation of blood on ARIO's parts additional measures can be implemented. One is coating the surface of ARIO parts with anti coagulant material. The second is to add anti-coagulant materials, such as but not limited to, heparin to the infusion liquid.

A second advantage is that the liquid that fluid flow in the concentric passage 10 will reduce the friction between the stationary outer tube 11 and the reciprocating inner tube 12. This will ease the operation of the whole system and will make the motion of the cutter smoother. That is to say, the passage of infusion fluid through the passageway configured in the atherectomy system brings the infusion fluid into contact with at least a two components of the operational mechanism of the ARIO system, wherein a relative position of the components one to another is varied during operation. It is to be noted that the friction reduction by the liquid is in addition to other means to reduce friction by coating the outer diameter of the inner tube and/or the inside diameter of the outer tube with low friction materials such as, but not limited to, Teflon. Reducing of friction is also applicable to the parts of ARIO's mechanism such as, but not limited to, the balls and groove that are part of the rotation generating arrangement of the ARIO system. The liquid is forced to pass in the space created between the distal piston 18 and the distal cylinder 19, and then flows in the groove 21 and over the balls 20, thus lowering the friction between these parts.

The third advantage of using the concentric lumen for the fluid flow path is that the lumen of the infusion liquid does not compete with the balloon lumens on space in the outer tube wall. (ARIO includes an option of using six balloons, i.e., there are six lumens in the outer tube). On the other hand, the concentric passage between the two tubes is an inherent part of ARIO's design.

The forth advantage is that the area of the concentric lumen in ARIO is larger than that of the lumen in the outer tube. As a result of that, the fluid flow rate via the concentric lumen is larger than the fluid flow rate in the lumen in the outer tube.

The fifth advantage is that the liquid in the concentric lumen is injected into the blood vessel substantially symmetrically relative to the axis of the blood vessel. Therefore, the flow of the liquid in all cutter slots is theoretically equal. In the former teaching the flow injected from the lumen located in the outer tube is not symmetrical.

The second feature of the infusion/suction system relates to the way the control system maintains the balance between fluid delivery and fluid removal. Prior art describes iso-volumetric systems i.e., the volume of the liquid entering and the flow leaving the artery are equal. The ARIO system controls the flow of infusion liquid and vacuum flow based on comparing the weight of the liquids. The system monitors and compares constantly the weight of liquid removed to the weight of liquid added to the body. Theoretically the weight method has a disadvantage when compared to the volume method. There is a difference between the specific gravity of the infusion liquid (saline) and the liquid leaving the body (a mixture of blood, saline and debris). However the difference is small. The specific gravity of saline is approximately 1.02 whereas blood has a specific gravity of approximately 1.06. That means the maximum difference in volumes is of approximately 4%. Usually this difference is smaller as the leaving liquid is a mixture of blood and saline. The body can tolerate this deviation. On the other hand implementation of the weight method is sterile, simpler to implement and cheaper than the iso-volumetric method that uses flow meters.

The system is sterile. All the components that come in contact with fluids are disposable. The cost of this system is relatively cheap. Load cells are much cheaper than non-contact flow meters (e.g., ultrasound or calorimetric flow meters). The load cells can also be used for giving the physician alarms in case infusion bag is empty or the collecting bottle is full.

An additional feature of the infusion and suction system relates to the suction side. In cases that debris is blocking the passage in the cutter or the inner tube, a flow regulator may be operated abruptly (totally shut down and fully open) to release the blockage.

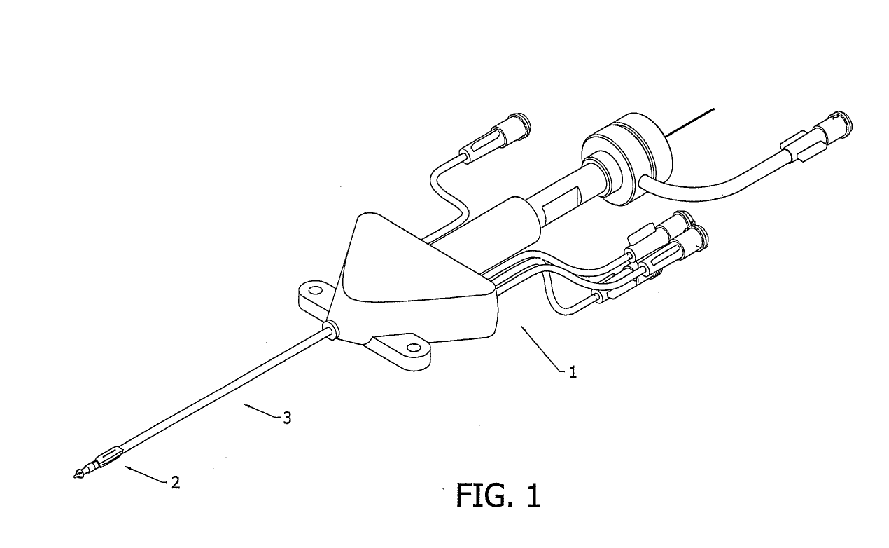

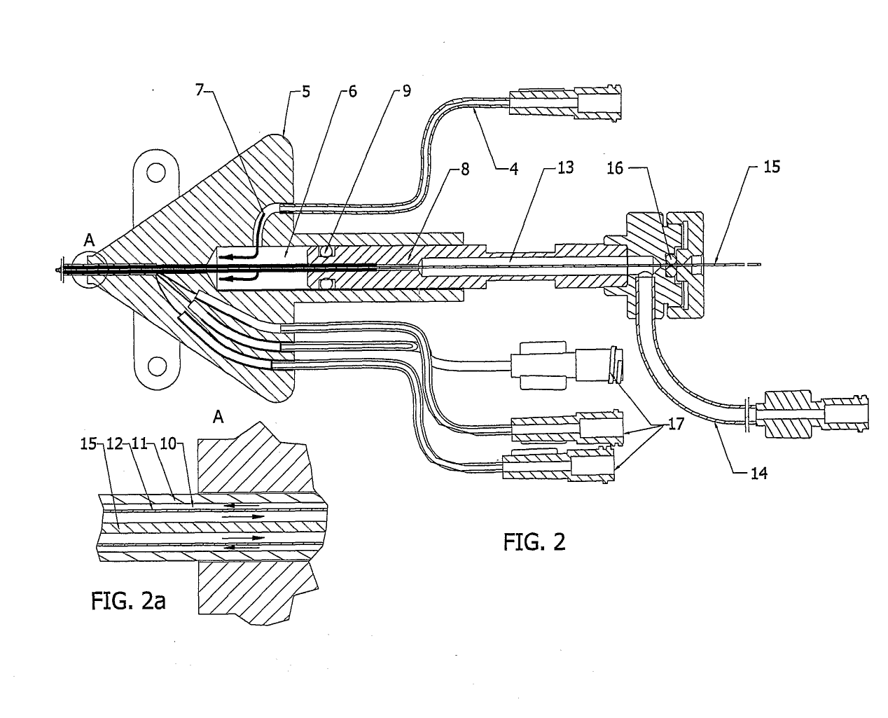

Referring now to the drawings, FIG. 1 is a general view of an ARIO catheter. The catheter comprises a proximal end 1 that is described in details in FIG. 2, a distal end 2 that is described in details in FIG. 3 and tubing 3 that connect the distal and proximal ends. The tubing is described in greater detail with regard to FIGS. 2 and 3

FIGS. 2 and 2a show the manner in which the infusion tube 4 is connected to proximal cylinder 5. Liquid flows via passage 7 into cavity 6. From cavity 6 the liquid can flow only distally as shown by the arrows. Proximal piston 8 has an O-ring 9 that seals cavity 6 proximally. FIG. 2a shows that the liquid flows distally in a concentric passage 10 that is formed between stationary outer tube 11 and reciprocating inner tube 12. The liquid reduces the friction created between the reciprocating inner tube 12 and stationary outer tube 11, which smoothes the operation of ARIO. The vacuum side of the ARIO system is also shown.

As illustrated here in FIG. 2, the inner tube 12 is connected to cavity 13 of proximal piston 8. Debris, excised from the atheroma, mixed with infusion liquid and blood are transferred proximally inside the inner tube to cavity 13 and from there towards a vacuum pump via vacuum tube 14. A guide wire 15 is located inside inner tube 12. Cavity 13 is sealed by O-ring 16 from the environment. Balloons tubes 17 that are used to inflate/deflate ARIO's balloons are shown here for reference purposes only.

FIG. 3 depicts the liquid circulation in the distal end of the ARIO system. The infusion liquid that arrives from the proximal end of ARIO in concentric passage 10 flows into concentric passage 17 that is created between distal piston 18 and distal cylinder 19. While flowing in passage 17, the infusion liquid flows over balls 20 and inside groove 21. A liquid with lubricity characteristics will ease the movement of balls 20 in groove 21 and thus ease the operation of ARIO. The infusion liquid pressure in passage 17 is substantially higher than the blood pressure, therefore blood generally cannot enter ARIO's mechanism. Following exit from passage 17 the liquid flows around cutter 22 and then is sucked out by the vacuum via cutter slots 23 to the inner tube 12. The liquid flowing via the cutter slots 23 is mixed with debris excised by the cutter and with blood.

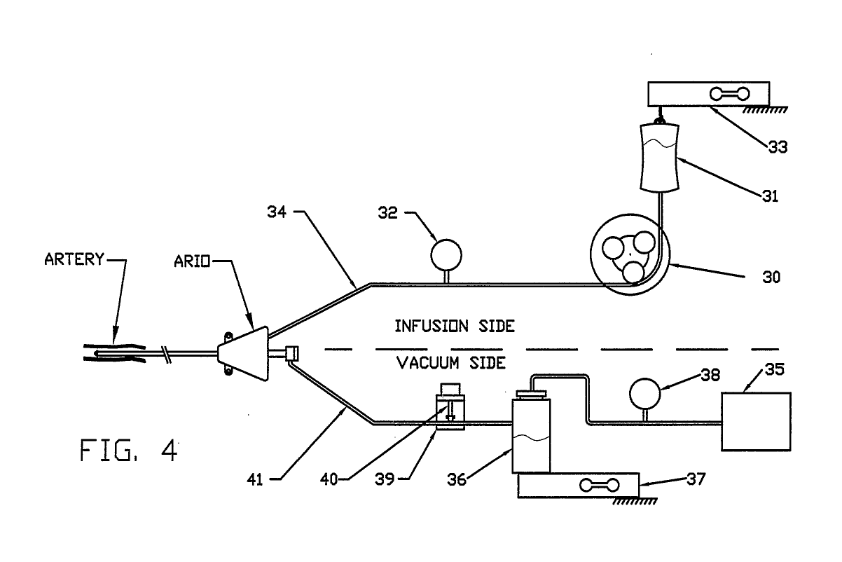

FIG. 4 schematically illustrates ARIO's infusion and suction system. The infusion side includes a peristaltic pump 30 that pumps infusion liquid such as, but not limited to, saline and heparin from an infusion bag 31. A pressure transducer 32 can optionally be added to monitor the infusion liquid pressure. The infusion bag 31 is suspended from a load cell 33. The load cell is electrically connected to the computerized control system (not shown in FIG. 4). The infusion tube 34 is connected to ARIO's infusion tube 4 (see FIG. 2). It is to be noted that all the units shown, except the infusion bag and the effluent collecting bottle, are connected to the control system. In this way the control system monitors constantly the weight of saline entering the artery.

The vacuum side of the ARIO system includes a vacuum pump 35 that is connected to an effluent collecting bottle 36. The effluent collecting bottle 36 is mounted on a load cell 37. Thus the weight of liquid leaving the artery can be constantly monitored by the computerized control system. A vacuum transducer 38 may be optionally added to the vacuum side. An electrical flow regulator 39 controls the flow of liquid on the vacuum side. The flow regulator 39 of the embodiment shown is, by non-limiting example, an electrical linear actuator 40. The linear actuator controls the flow by squeezing the vacuum tube 41 from the outside, thus there is no physical connection between the components of the regulator 39 and the fluid. This is important for keeping the infusion/suction system components sterile. The vacuum tube 41 is connected to ARIO's vacuum tube 14 (see FIG. 2).

The procedure of the computerized control system begins with measuring the weight of both the infusion bag 31 and the effluent collecting bottle 36. It will be appreciated that these weights may vary, but it has no significance to the system as the computerized control system monitors only the differences between the weight added to the effluent collecting bottle 36 and the reduction in weight of the infusion bag 31. That is to say, the control system uses a weight comparison method in which the relative weight difference between the infusion bag and the effluent collecting bottle are compared. The algorithm of the control system is to minimize the deviation in the weights. The peristaltic pump 30 and the flow regulator 39 regulate the liquid flow in the infusion side and in the vacuum side respectively.

It will be appreciated that the above descriptions are intended only to serve as examples and that many other embodiments are possible within the spirit and the scope of the present invention.

What is claimed is:

1. A method for expelling blood and preventing blood from entering portions of an atherectomy system, the method comprising the passage of infusion fluid through a passageway configured in the atherectomy system, said passageway configured to provide a flow of said infusion fluid through an operational mechanism of the atherectomy system and exit the atherectomy system at a higher pressure and higher velocity than that of blood flow within a blood vessel in which the atherectomy system is being operated.

2. The method of claim 1, further including implementing said passageway between an inside wall of a stationary outer tube and an outside wall of an inner displaceable tube.

3. The method of claim 1, further including directing said infusion fluid through at least a portion of a rotation generating arrangement of the atherectomy system.

4. The method of claim 1, further including lubricating portions of the atherectomy system, by bringing said infusion fluid into contact with at least two components of an operational mechanism of the atherectomy system, wherein a relative position of said at least two Components one to another is varied during operation of the atherectomy system.

5. An atherectomy system comprising a passageway configured to bring an infusion fluid into contact with at least a two components of an operational mechanism of the atherectomy system, wherein a relative position of said at least two components one to another is varied during operation of the atherectomy system.

6. The atherectomy system of claim 5, wherein said at least two components are substantially adjacent one to another.

7. The atherectomy system of claim 5, wherein said passageway is configured between an inside wall of a stationary outer tube and an outside wall of an inner displaceable tube.

8. The atherectomy system of claim 5, wherein said passageway is configured so as to direct said infusion fluid through at least a portion of a rotation generating arrangement of the atherectomy system.

9. The atherectomy system of claim 5, further including a pump arrangement configured to provide a fluid flow through said passageway at a higher pressure and higher velocity than that of blood within a blood vessel in which the atherectomy system is being operated so as to expel blood from said operational mechanism by.

10. An atherectomy system comprising a control system that monitors and regulates a flow of fluid infused into a blood vessel and a flow of fluid leaving said blood vessel by a weight comparison method in which a relative weight difference between an infusion bag and an effluent collecting bottle are compared.

11. The atherectomy system of claim 10, further including at least a first load cell from which said infusion bag is suspended and at least a second load cell upon which said effluent collecting bottle is mounted.

12. The atherectomy system of claim 10, further including at lease a first pumping arrangement for introducing infusion fluid into the atherectomy system and at least a second pumping arrangement for evacuate of a mixture of debris, blood and infusion fluid from the atherectomy system, wherein said first and second pumping arrangements are regulated by said control system based on said relative weight difference between said infusion bag and said effluent collecting bottle.
